SEQUENCE LISTING (9 sequences)

SEQ ID NO: 1 - DNA, 1140 bp, Trypanosoma cruzi
atggcgacgttccctgaacttctgcggcccctcaaattggggcgctacacacttcgtaatcggattattatggctcccttgacgcgttgccaggcaacagaagatgatcatgtaccaaggacggaatcgatgctgaagtactacgaagaccgggcatctgcaggtcttatcattgccgaggcgacgatggtccagccaaactacactgggttcctcacggagcctggcatttactccgatgcgcagattgaggagtggagaaagatcgtggacgcggtacacaaaaagggtggccttatattcctgcaactcattcacgctggtcgagccgggattccggagaagatccttcagcagtcgaagagtgaccaggatccccttgctgggcgcctgcttgccgcgagtgccattcccattaaggaccatcggattcctgcctattttgctgcgagcggagaaaaggagacctacggtgtcccagaggagctcacggatgacgaagtccgggacggtatcatcccattgtttgtggagggggccaaaaacgccatctttaaggctgggtttgatggcgttgagattcatggagccaacggctacttactggacgccttttttcgcgaatcttccaacaagcgccagtccggtccgtacgccggaacgaccatcgacacacgatgccaactcatctacgatgtcaccaaaagcgtctgcgatgccgtgggaagtgaccgtgtggggctccgcatatccccactaaacggcgtgcatgggatgattgactcgaacccggaggcactaaccaagcatctatgcaagaaaattgagccactttcgcttgcctatctgcattacttgcgtggcgacatggtcaaccagcagattggtgacgttgtggcgtgggttcgtggaagttacagcggtgtaaaaatatccaacttgcgctacgatttcgaagaggcagaccagcaaatacgggaaggaaaagtcgacgccgtggcttttggcgccaagttcattgcgaaccccgatctcgttgaaagggcccaacaaaactggcccctcaacgagccgcgaccagaaacatactacacaagaacagcagtcggatacaacgattacccgacgtacaacaaataa

SEQ ID NO: 2 - protein, 379 aa, Trypanosoma cruzi
MATFPELLRPLKLGRYTLRNRIIMAPLTRCQATEDDHVPRTESMLKYYEDRASAGLIIAEATMVQPNYTGFLTEPGIYSDAQIEEWRKIVDAVHKKGGLIFLQLIHAGRAGIPEKILQQSKSDQDPLAGRLLAASAIPIKDHRIPAYFAASGEKETYGVPEELTDDEVRDGIIPLFVEGAKNAIFKAGFDGVEIHGANGYLLDAFFRESSNKRQSGPYAGTTIDTRCQLIYDVTKSVCDAVGSDRVGLRISPLNGVHGMIDSNPEALTKHLCKKIEPLSLAYLHYLRGDMVNQQIGDVVAWVRGSYSGVKISNLRYDFEEADQQIREGKVDAVAFGAKFIANPDLVERAQQNWPLNEPRPETYYTRTAVGYNDYPTYNK

SEQ ID NO: 3 - protein, 10 aa, Trypanosoma cruzi
NRIIMAPLTR

SEQ ID NO: 4 - protein, 10 aa, Trypanosoma cruzi
DHRIPVYFAA

SEQ ID NO: 5 - protein, 10 aa, Trypanosoma cruzi
ISNLRYDFEE

SEQ ID NO: 6 - DNA, 30 bp, Trypanosoma cruzi
cggaattcatggcgacgttccctgaacttc

SEQ ID NO: 7 - DNA, 30 bp, Trypanosoma cruzi
ccgctcgagttatttgttgtacgtcgggta

SEQ ID NO: 8 - DNA, 22 bp, Trypanosoma cruzi
atggcgacgttccctgaactcc

SEQ ID NO: 9 - DNA, 21 bp, Trypanosoma cruzi
ttatttgttgtacgtcgggta

(19) United States
(12) Patent Application Publication
(10) Pub. No.: US 2006/0275329 A1
(43) Pub. Date: Dec. 7, 2006

(54) Flavin protein of trypanosoma cruzi, method of screening vermicide with the use of the same and diagnostic

(75) Inventors: Yoshihiro Urade, Kyoto-fu (JP); Bruno Kilunga Kubata, Nairobi (KE); Pius Zakayi Kabututu, Osaka-fu (JP); Tomoyoshi Nozaki, Tokyo-to (JP)

Correspondence Address: BROWDY AND NEIMARK, P.L.L.C.;624 NINTH STREET, NW, SUITE 300, WASHINGTON, DC, 20001-5303, US

(73) Assignees: Osaka Bioscience Institute, Suita-shi (JP); JAPAN AS REPRESENTED BY THE DIRECTOR-GENERAL OF NATIONAL INSTITUTE OF INFECTIOUS DISEASE, Shinjyuku-ku (JP)

(21) Appl. No.: 10/530,865

(22) PCT Filed: Oct. 10, 2003
(86) PCT No.: PCT/JP03/13043

§ 371 Date: Apr. 11, 2005

(30) Foreign Application Priority Data
Nov. 10, 2002 (JP) 2002-299284

(51) Int. Cl. C12Q 1/68; C07H 21/04; A61K 39/00; C12Q 1/26; C12N 9/02; C07K 16/20

(52) U.S. Cl. 424/269.1; 530/388.6; 435/189; 536/23.2; 435/258.1; 435/6; 435/25

(57) ABSTRACT

It is intended to provide a method of diagnosing infection with Chagas disease by screening a trypanocidal drugs for Trypanosoma cruzi which is the pathogen of Chagas disease. Using a flavin protein TcOYE specific to Trypanosoma cruzi, a trypanocidal drugs effective against Trypanosoma cruzi is screened. Using the gene sequence of TcOYE and an antibody therefor, infection with Trypanosoma cruzi is diagnosed.

### TECHNICAL FIELD

The present invention relates to the development of an effective trypanocidal drug for treating Trypanosoma cruzi infection (Chagas' disease) for which no effective treatment is currently available; and to a simple, highly specific diagnostic method. More particularly, the present invention relates to a method of developing a trypanocidal drug effective against Trypanosoma cruzi, the pathogen of Chagas' disease, using the flavoprotein TcOYE present in Trypanosoma cruzi and its recombinant protein; and of testing for the metabolic rate (degradation activity) of the effective substance. Furthermore, the invention relates to a simple and specific method of diagnosing Trypanosoma cruzi infection using the gene sequence of TcOYE and antibodies thereto.

### BACKGROUND ART

Chagas' disease is a parasite infection caused by Trypanosoma cruzi (World Health Organization, Weekly Epidemiol. Res. 65: 257-264, 1990; Coura J. R. et al., Trends Parasitol., 18: 171-176, 2002; TeiXeira, M. M. et al., Trends Parasitol., 18: 262-268, 2002). This protozoa infects various animals other than humans, such as dogs, cats, and armadillo, and is distributed in many states of the US and all over Central and South America. Infection of human occurs mainly in areas south of Texas and has been reported in 18 countries of Central and South America. The number of cases is estimated at 16 to 18 million, resulting in 21,000 deaths every year, and increasing by 300,000 every year. There are 2,000,000 to 3,000,000 infected patients in the chronic stage, and people in areas at a high risk for infection amount to 120,000,000.

Trypanosoma cruzi, when present in human blood, has large kinetoplasts and is of the trypomastigote type with a C-shaped curved body 18 to 22 μm in length, and it undergoes no division or multiplication. However, in cells of the muscles, the liver, the spleen, and the heart, it is of the a trichous type with a slightly elliptic body 2 to 4 μm in diameter, having large kinetoplasts, and it multiplies by binary fission. This atrichous type can develop into the epimastigote and promastigote types, but it finally develops into the trypomastigote type. When this organism is sucked by the vector assassin bugs, it goes through the a trichous type into the metacyclic Trypanosoma type in the body, and is excreted in the feces. It requires about 10 days for development in the insect body.

The vector assassin bug is a relatively large size insect and divided into many known species, and examples of important species include Triatoma infestans which is distributed over the southern part of South America including Argentina, Rhodnius prolixus distributed over the northern part of South America, and Central America, and Panstrongylus megistus in Brazil. When a person is stung by such insect, it causes severe pain and itching, and infection occurs when the person scratches the sting wound and the Trypanosoma in the insect feces excreted on the skin is rubbed into the wound. An assassin bug occurs frequently in a house, and sucks blood at night. Male and female adults, young insects and larvae can be a vector.

When stung by an insect and infected with Trypanosoma cruzi, a red boss called chagoma forms at the site. Symptoms appear after a one to two-week latent period. Acute symptoms occur mainly in infants. Such conditions as high fever, eruption, lymphadenitis, hepatosplenomegaly, facial and unilateral, in particular, blepharedema called Romana symptom, myocarditis and meningoencephalitis may occur and sometimes result in death after a disease progress over 2 to 4 weeks. An infant patient who has passed through an acute stage shifts to a chronic stage, while an adult patient tends to go through a chronic course from the beginning. Examples of cardinal symptoms during the chronic stage include myocarditis, cardiac hypertrophy and giant colon.

Diagnosis of Chagas' disease includes, starting with checking for the characteristic symptoms mentioned above, morphological detection of the protozoa using smear/Giemsa stained specimens from blood and lymph node aspiration, the protozoan culture detection method using synthetic culture medium, the animal inoculation method which involves injecting the sampled material into rats or mice so as to multiply in the body, the vector diagnostic method which involves allowing uninfected assassin bugs to suck blood from a patient then detecting the protozoa that has multiplied in the insect intestine two weeks later, or immunological diagnostic methods such as endodermal reaction, complement fixation reaction, and fluorescent antibody technique. However, any of these methods requires skill and complex procedures, posing sensibility and specificity problems.

Nifurtimox and benznidazole have been used as a treatment for Chagas' disease (Docampo, R. & Moreno, S. N. S., FASEB J. 45, p 2471-2476, 1986; Henderson, G. B., et al., Proc. Natl. Acad. Sci. USA, 85, p 5374-5378, 1988; Docampo, R., Chem. Biol. Interactions, 73, p 1-27, 1990). However, these drugs are effective for only the early infection phase (Braga M. S., et al., Rev. Inst. Med. trop. S. Paulo, 42, p 157-161, 2000), and nothing is known about the mechanism of action except that the involvement of radicals such as reactive oxygen has been suggested (Boveris, A. R. et al., Biochem J., 175: 431-439, 1978; Boveris, A. et al., Comp. Biochem. Physiol., 61C: 328-329, 1978; Docampo, R. & Stoppani, A. O., Arcch. Biochem. Biophys., 197: 317-321, 1979; Viode, C. N. et al, Biochem. Pharmacol., 57: 549-557, 1999). Besides, they cause strong adverse effects and are carcinogenic. The antimalarial primaquine is thought to be effective to some extent, but other drugs which are effective in treating African trypanosomiasis and leishmaniasis are ineffective against Chagas' disease and no vaccine for Trypanosoma cruzi has been developed. Thus there is no effective drug for treatment of Chagas' disease. Therefore, there is a need worldwide to search for a new target molecule for the development of drugs to address Chagas' disease (World Health Organization TDR news 67: 15, 2002).

Meanwhile, the inventors have indicated the possibilities that parasitic protozoan such as malarial parasite and Trypanosoma brucei, the pathogen of African trypanosomiasis, have a metabolic system to biosynthesize prostaglandins (PGs), which possess sleep inducing, vasodilating and immunosuppressing actions, from arachidonic acid, that the PG synthesis system in these protozoans is irresponsive to inhibitors of the mammalian enzyme (cyclooxygenase), and that these protozoans may use PGs to establish parasitism in the host (Kubata B. K. et al., J. Exp. Med. 188: 1197-1202, 1998; Kubata B. K. et al., J. Exp. Med. 192: 1327-1337, 2000). Then, the inventors purified from the soluble fraction of Trypanosoma brucei all lysates, Trypanosoma brucei PGF synthase enzyme (TbPGFS), which reduces prostaglandin H₂ (PGH₂), the common precursor of various PGs, to prostagland in F_{2α} (PGF_{2α}) and cloned the gene and cDNA thereof, demonstrating that TbPGFS belongs to the aldoketo reductase gene family (Kubata B. K. et al., J. Exp. Med. 192: 1327-1337, 2000). However, it was not made clear whether or not Trypanosoma cruzi, which forms a cytozoon, has a PG biosynthetic pathway as malarial parasite and Trypanosoma brucei, which have different infection routes and taxonomical positions.

### DISCLOSURE OF THE INVENTION

(Problems to be Solved by the Invention)

It is an object of the present invention to provide a new target molecule for the development of drugs for Chagas' disease as well as to provide a method of screening therapeutic drugs for Trypanosoma cruzi infection and a diagnostic.

The inventors have conducted intensive studies to achieve the object, and completed the invention based on the findings as follows:

1) Trypanosoma cruzi also has the metabolic system that biosynthesizes prostaglandin (PG) from arachidonic acid, and the PG synthetic system of this protozoan is not inhibited by inhibitors of the mammalian enzyme (cyclooxygenase). It is very likely that Trypanosoma cruzi also uses PG to establish parasitism in the host as Trypanosoma bruci does.

2) The soluble fraction of Trypanosoma cruzi has an enzymatic activity to reduce PGH₂ to PGF_{2α} in the presence of NADPH or NADH, and the activity is not absorbed by an antibody to the Trypanosoma PGF synthetic enzyme (TbPGFS). Since the T. cruzi PGF_{2α} synthesizing protein has not been detected in other protozoan parasites, such as Trypanosoma brucei and leishmania, or mammals including humans, it is thought to be peculiar to Trypanosoma cruzi.

3) The PGH₂-PGF_{2α} reductase purified from the Trypanosoma cruzi soluble fraction is a flavoprotein containing equimolar FMN.

4) cDNA of the protein has a protein translation region of 1,140 base pairs, and encodes a protein of molecular weight of 42,260 consisting of 379 amino acid residues.

5) From homology analysis based on the predicted amino acid sequence, this enzyme belongs to the old yellow enzyme (NADPH dehydrogenase) gene family, which is absent in animals. Thus, this enzyme was designated TcOYE.

6) The recombinant TcOYE produced and purified in large quantities through expression in E. coli using the cDNA exhibits a PGH₂-PGF_{2α} reductase activity with specific activity similar to that of the enzyme purified from the soluble fraction of Trypanosoma cruzi. Under anaerobic condition, the recombinant TcOYE catalyzes the reduction reaction of compounds lethal to Trypanosoma, such as hydrogen peroxide and butyl peroxide, as well as menadione, β-lapachone, nifurtimox, and 4-nitroquinoline-N-oxide.

7) TcOYE converts naphtoquinone compounds such as menadione and β-lapachone to semiquinone radicals by one-electron reduction. On the other hand, when the substrate is nifurtimox or 4-nitroquinoline-N-oxide, TcOYE conducts two-electron reduction and does not produce radicals.

8) Polyclonal antibodies to the TcOYE immunoprecipitate almost completely the activity of the PGH₂-PGF_{2α} reductase present in the soluble fraction of Trypanosoma cruzi and the activity to reduce such compounds as menadione, β-lapachone, nifurtimox and 4-nitroquinoline-N-oxide.

Since TcOYE has not been found in other protozoan parasites, such as Trypanosoma brucei and leishmania, or mammals including humans, it may provide a good target for the development of trypanocidal drugs specific for Trypanosoma cruzi. In a screening of an enzymatic reaction using the recombinant TcOYE, the compounds that will undergo one-electron reduction to produce radicals are expected to have an antiparasitic effect against Trypanosoma cruzi, while the compounds that will undergo two-electron reduction are expected to be decomposed easily by Trypanosoma cruzi.

In addition, detection of the TcOYE protein and gene by an immunological method using antibodies to TcOYE and a molecular biological method using the nucleotide sequence of the TcOYE gene, such as the RT-PCR method, can be applied in developing a highly specific and simple method for diagnosing Trypanosoma cruzi infection.

(Solutions)

The present invention relates to the flavoprotein TcOYE derived from Trypanosoma cruzi, having the enzymatic activity to reduce prostaglandin H₂ to prostaglandin F_{2α}.

In one aspect, the present invention relates to the recombinant protein of (a), (b) or (c) below:

(a) a protein containing the amino acid sequence represented by SEQ ID NO: 2

(b) a protein comprising an amino acid sequence having deletion, substitution or addition of one or more amino acids in the amino acid sequence represented by SEQ ID No: 2, and having an enzymatic activity to reduce prostaglandin H₂ to prostaglandin F_{2α}

(c) a protein comprising a fragment of the amino acid sequence represented by SEQ ID NO: 2, and having an enzymatic activity to reduce prostaglandin H₂ to prostaglandin F_{2α}.

The present invention further relates to the gene encoding the protein described above.

In one aspect, the present invention relates to the gene above consisting of the DNA containing the base sequence of SEQ ID NO: 1.

The present invention further relates to an antibody to the protein described above.

The present invention further relates to a method of screening for trypanocidal drugs against Trypanosoma cruzi infection, comprising the steps of:

(i) preparing the protein described above and prostaglandin H₂,

(ii) contacting the protein and prostaglandin H₂ with a candidate compound in the presence of NADPH or NADH, and

(iii) examining whether or not the reduction of prostaglandin H₂ to prostaglandin F_{2α} is inhibited.

In another aspect, the present invention further relates to a method of screening trypanocidal drugs against Trypanosoma cruzi infection, comprising the steps of:

(i) incubating the protein described above with a candidate compound in the presence of NADPH or NADH, and

(ii) determining whether or not the compound produces radicals as a result of one-electron reduction by the protein.

The present invention further relates to a method of diagnosing Trypanosoma cruzi infection, comprising the steps of:

(i) incubating a specimen or the extract of a specimen with the antibody described above, and

(ii) examining whether or not an antigen/antibody complex forms.

In another aspect, the present invention relates to a method of diagnosing Trypanosoma cruzi infection, comprising the steps of:

(i) reacting a specimen or the extract of a specimen with the gene described above or a fragment thereof, and

(ii) examining whether or not they hybridize to each other.

In another aspect, the present invention relates to a method of diagnosing Trypanosoma cruzi infection, comprising the steps of:

(i) preparing DNA collected from a specimen or cDNA synthesized from mRNA in a specimen using reverse transcriptase,

(ii) performing PCR using this DNA or cDNA as a template, and using the nucleotide sequence contained in the cDNA of TcOYE of SEQ ID NO: 1 as a sense primer and an antisense primer,

(iii) examining whether or not the cDNA of TcOYE is amplified.

### BRIEF DESCRIPTION OF THE DRAWINGS

FIG. 1 is a graph showing production of prostaglandin by the crude extract of Trypanosoma cruzi;

FIG. 2 is a view of a silica gel thin layer chromatograph detecting [1-14c]-PGF_{2α} from the reduction of [1-14c]-PGH₂ in the presence of NADPH in the Trypanosoma cruzi soluble fraction;

FIG. 3 is a view of an SDS polyacrylamide gel electrophoresis profile of the purified enzyme from Trypanosoma cruzi showing a uniform band at molecular weight of 42,000;

FIG. 4 is a table showing the yield and purification ratio by the purification steps of the prostaglandin H₂-F_{2α} reductase from Trypanosoma cruzi;

FIG. 5 is an absorption spectrum in the visible region of the oxidation-state PGH₂-F_{2α} reductase purified from Trypanosoma cruzi;

FIG. 6 is a schematic view showing expression of the recombinant TcOYE and an SDS polyacrylamide gel electrophoresis profile of the specimen in different purification steps;

FIG. 7 is a table showing the substrate specificity of reduction reaction by the recombinant TcOYE;

FIG. 8 is a view showing electron spin resonance spectra of semiquinone radicals produced through one-electron reduction of naphtoquinone compounds by TcOYE and superoxide anion radicals produced through secondary reaction with oxygen;

FIG. 9 is a graph showing inhibition of the PGH2-F2α reductase activity of TcOYE by naphtoquinone compounds and nitro hetero-cycle compounds;

FIG. 10 is a view of Western blot assay using a crude extract of Trypanosoma indicating the specificity of anti-TcOYE antibody;

FIG. 11 is a schematic view of a silica gel thin layer chromatograph showing immunoabsorption of the PGH₂-F_{2α} reductase activity in the Trypanosoma cruzi soluble fraction by an anti-TcOYE antibody; and

FIG. 12 is a table showing immunoprecipitation by the anti-TcOYE antibody for the enzymatic activity to reduce menadion, β-lapachone, nifurtimox, 4-nitroquinoline-N-oxide in the crude extract of Trypanosoma cruzi.

### DETAILED DESCRIPTION OF THE INVENTION

The present invention relates to the flavoprotein TcOYE derived from Trypanosoma cruzi, having the enzymatic activity to reduce prostaglandin H₂ to prostaglandin F_{2α} in the presence of NADPH or NADH.

The present invention further relates to the recombinant protein of (a), (b) or (c) below:

(a) a protein containing the amino acid sequence represented by SEQ ID NO: 2

(b) a protein (hereinafter sometimes referred to as “variant TcOYE”) comprising an amino acid sequence having deletion, substitution or addition of one or more amino acids in the amino acid sequence represented by SEQ ID No: 2, and having an enzymatic activity to reduce prostaglandin H₂ to prostaglandin F_{2α}

(c) a protein (hereinafter sometimes referred to as “TcOYE fragment”) containing a fragment of the amino acid sequence represented by SEQ ID NO: 2, and having an enzymatic activity to reduce prostaglandin H₂ to prostaglandin F_{2α}.

This protein (TcOYE) has the following properties.

(1) It contains equimolar flavin mononucleotide (FMN), and has a molecular weight of about 42,000.

(2) It has an enzymatic activity to reduce PGH₂ to PGF_{2α} in the presence of NADPH or NADH.

(3) It also reduces hydrogen peroxide, butyl peroxide, menadion, β-lapachone, 4-nitroquinoline-4-oxide, nifurtimox, phenazine methosulfate (5-methyl-phenazium methyl sulfate), mevinolin (2β,6α-dimethyl-8α-(2-methyl-1-oxo-butoxy)-mevinic acid lactone), 12-oxo-phytodienoic acid (4-oxo-5β-(2Z-pentenyl)-2-cyclopentene-lp-octanoic acid), 9-oxo-10E,12Z-octadecadienoic acid, econazole(1-[2-([4-chlorophenyl]methoxy)-2-(2,4-dichlorophenyl)ethyl-1H-imidazole)].

(4) Reduction reactions include one-electron reduction where the substrate produces radicals and two-electron reduction where no radicals are formed.

(5) The enzymatic activity to reduce PGH₂ to PGF_{2α} is absorbed completely by the anti-TcOYE antibody.

(6) The enzymatic activity to reduce PGH₂ to PGF_{2α} is not absorbed by the anti-TbPGFS antibody.

The inventors succeeded in cloning the cDNA encoding the protein TcOYE from Trypanosoma cruzi. The cDNA has the base sequence of SEQ ID NO: 1. Therefore, TcOYE has the predicted amino acid sequence of SEQ ID NO: 2.

This protein (TcOYE) can be produced through isolation from Trypanosoma cruzi, but it is preferable to produce the protein using genetic recombination technology.

Procaryotes can be used for producing the protein of the present invention. Examples of procaryotes suitable for the production of the protein of the present invention include Escherichia coli K-12 294 and other E. coli strains, bacillus species such as Bacillus subtilis, intestinal bacteria such as Salmonella typhimurium and Seratia marcescans, various Pseudomonas species and Streptomyces species.

Examples of promoter sequences suitable for controlling gene expression in procaryotes include β-lactamase, lactose, alkaline phosphatase and tryptophan (trp) promoters. Hybrid promoters such as tac promoter may also be suitable. Generally, other bacterial promoters with known base sequences may be linked to the DNA encoding the protein of the present invention by use of a linker or an adapter that provides any necessary restriction site.

The term “gene” as used herein refers to any molecule, such as DNA or RNA, having a nucleic acid sequence with the sequence described above.

The present invention relates also to a vector, and especially a plasmid, cosmid, virus, bacteriophage and other conventional vectors used in genetic engineering that contains the nucleic acid of the present invention. Various plasmids and vectors can be created using methods known to those skilled in the art. See for example, Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1989), (1994). Plasmids and vectors preferably used according to the present invention include those known to those skilled in the art.

In a preferred embodiment, a nucleic acid molecule present in a vector is linked to the control sequence that can express the gene in procaryotic or eucaryocytic cells.

The term “control sequence” refers to a regulatory DNA sequence necessary for the expression of the coding sequence to which it is linked. Properties of such control sequence vary depending on the host organism. In procaryotes, a control sequence generally contains a promoter, a ribosome-binding site and a terminator. In eucaryotes, a control sequence generally contains a promoter, a terminator and in some cases a transactivator or a transcription factor. The term “control sequence” implies that at least all components necessary for expression are contained, and other useful components may be contained.

The term “operably linked” implies a position that allows the component to operate in the intended manner. A control sequence “operably linked” to a coding sequence means that it is linked in a manner such that the expression of the coding sequence may be achieved under the condition compatible to the control sequence. When the control sequence is a promoter, a double-stranded nucleic acid is preferably used, as is well known to those skilled in the art.

Therefore, preferably the vector according to the present invention is an expression vector. “Expression vector” is a construct that can be used to transform a selected host cell and express a coding sequence in the selected host cell. For example, the expression vector can be a cloning, binary or integrating vector. Preferably the expression includes transcription of a nucleic acid molecule to a translatable mRNA. Regulatory factors that ensure expression in procaryotic and/or eucaryotic cells are known to those skilled in the art. In the case of eucaryotes, such regulatory factors contain a promoter ensuring the initiation of transcription and, in some cases, a poly A signal ensuring the termination of transcription and the stabilization of transcripts. Promoters generally used are a polyubiquitin promoter and an actin promoter. Other regulatory factors may include a transcription enhancer. Possible regulatory factors enabling expression in procaryotic host cells include, for example, PL, lac, trp or tac promoters in E. coli. Examples of appropriate regulatory factors known to those skilled in the art which enable expression in eukaryotic host cells include AOX1 or GALL promoter in yeast, CMV- in mammalian and other animal cells, SV40-, RSV- (avian sarcoma virus) promoters, CMV enhancer, SV40 enhancer or the expression vector pcDV1 (Pharmacia) of Okayama-Berg which is globin intron, and the expression vectors pCDM8, pRc/CMV, pcDNA1, PcDNA3 (In-vitrogen) and pSPORT1 (GIBCO BRL). Another expression system which can be used to express the protein is an insect system. By one such system using Autographa california nuclear polyhedrosis virus (AcNPV) as a vector, a foreign gene is expressed in Spodoptera frugiperda cells or Trichoplusia larvae. The coding sequence of the gene of the present invention may be cloned into nonessential viral regions such as the polyhedrin gene, and placed under the control of the polyhedrin promoter. Successful insertion of the coding sequence would inactivate the polyhedrin gene and produce a recombinant virus lacking in a coat protein. The recombinant virus is used to infect Spodoptera frugiperda cells or Trichoplusia larvae, in which the protein of the present invention is expressed (Smith, J. Virol. 46 (1983), 584; Engelhard, Proc. Nat. Acad. Sci. USA 91 (1994), 3224-3227). Advantageously, the vector of the present invention described above contains a selectable marker.

The present invention further relates to a host cell containing the vector described above or the gene of the invention wherein the nucleic acid sequence is exogenous to the host cell.

The term “exogenous” implies that a nucleic acid molecule is either heterologous to a host cell (meaning that it is derived from a cell or organism having a different genetic background) or homologous to a host cell but it has a different genetic background from the natural counterpart of the nucleic acid molecule. This means that if the nucleic acid molecule is homologous to a host cell, it is not located at the natural site in the genome of the host cell and is surrounded, in particular, by different genes. In this case, the nucleic acid molecule may be under the control of the original promoter or a heterologous promoter. The vector or gene of the present invention contained in a host cell may be incorporated into the genome of the host cell or maintained outside the chromosome within the cell. In this regard, the gene of the present invention may be used to recover or create a variant gene by homologous recombination (ed. Paszkowski, Homologous Recombination and Gene Slicing in Plants, Kluwer Academic Publishers (1994)).

Thus, the present invention relates to a host cell containing the vector or gene of the present invention. The host cell may be any procaryotic or eucaryotic cell including bacteria (archaebacteria), insects, fungi, plants and animals. Preferably, the fungal cell is Saccharomyces species, and especially Saccharomyces cerevisae.

The term “procaryotic” is used to include all bacteria that can be transformed or transfected with DNA or RNA for the expression of the protein of the present invention. Procaryotic hosts can include, for example, gram-positive and gram-negative bacteria such as E. coli, S. typhimurium, Seratia marcescens and Bacillus subtilis. The term “eukaryotic” is used to mean cells of yeast, higher plants, insects, and preferably mammals. The protein encoded by the polynucleotide of the present invention may or may not be glycosylated by the host used for the production of a recombinant. The protein of the present invention may or may not have the first amino acid residue methionine. Any technique generally known to those skilled in the art may be used to transform or transfect a host with the gene of the present invention. Furthermore, methods for fusion, preparation of a functionally bound gene, and expression thereof in, for example, in mammals and bacteria are known to those skilled in the art (Sambrook, Molecular Cloning: A Laboratory Manual, Cold Spring Harbor Laboratory, Cold Spring Harbor, N.Y., 1989).

The present invention further relates to the gene encoding the protein of (a), (b) or (c) below:

(a) flavoprotein TcOYE derived from Trypanosoma cruzi, having an enzymatic activity to reduce prostaglandin H₂ to prostaglandin F_{2α} in the presence of NADPH or NADH.

(b) a protein containing the amino acid sequence represented by SEQ ID NO: 2

(c) a protein comprising an amino acid sequence having deletion, substitution or addition of one or more amino acids in the amino acid sequence represented by SEQ ID No: 2, and having an enzymatic activity to reduce prostaglandin H₂ to prostaglandin F_{2α}

(d) a protein containing a fragment of the amino acid sequence represented by SEQ ID NO: 2, and having an enzymatic activity to reduce prostaglandin H₂ to prostaglandin F_{2α}.

In one aspect, the gene encoding the protein having the amino acid sequence represented by SEQ ID NO: 2 consists of the DNA containing the base sequence of SEQ ID NO: 1. There can be many base sequences depending on how the genetic code is degenerated.

The invention further relates to an antibody to:

(a) a protein containing the amino acid sequence represented by SEQ ID NO: 2

(b) a protein (hereinafter sometimes referred to as “variant TcOYE”) comprising an amino acid sequence having deletion, substitution or addition of one or more amino acids in the amino acid sequence represented by SEQ ID No: 2, and having an enzymatic activity to reduce prostaglandin H₂ to prostaglandin F_{2α}, or

(c) a protein containing a fragment of the amino acid sequence represented by SEQ ID NO: 2, and having an enzymatic activity to reduce prostaglandin H₂ to prostaglandin F_{2α}.

These proteins can be used as a source of immunity to produce an antibody thereto. These antibodies can be polyclonal or monoclonal antibodies. The present invention also includes chimera, single chain and humanized antibodies as well as Fab fragments or the products of a Fab expression library. Various methods known in the art can be used for the production of such antibodies and fragments.

Antibodies generated against the protein corresponding to the sequence of the present invention can be obtained by injecting the protein directly into an animal or administering the protein to an animal, preferably non-human animal. The anti-TcOYE antibody obtained in such manner binds to TcOYE itself, and absorbs completely the PGH₂-PGF_{2α} reductase activity. Using this method, even a sequence encoding a fragment of a protein can be used for producing an antibody binding to the complete natural protein.

Any technology providing an antibody produced by continuous cell culture can be used to prepare a monoclonal antibody. Examples of such technologies include hybridoma technology (Kohler and Milstein, 1975, Nature, 256: 495-497), trioma technology, human B cell hybridoma technology (Kozbor et al., 1983, Immunology Today 4: 72) and EBV-hybridoma technology to produce human monoclonal antibody (Cole et al., 1985, Monoclonal Antibodies and Cancer Therapy, Alan R. Liss, Inc., 77-96).

The present invention relates to a method of screening trypanocidal drugs against Trypanosoma cruzi infection, comprising the steps of:

(i) preparing the protein TcOYE and prostaglandin H2, (ii) incubating the protein and prostaglandin H2 with a candidate compound in the presence of NADPH or NADH, and

(iii) examining whether or not the reduction of prostaglandin H2 to prostaglandin F2α is inhibited.

An example of the reaction is as follows. For reaction under aerobic condition, 1 μl of 500 μM [1-14C]PGH₂ solution (2.04 Gbq/mmol; acetone, DMSO or dimethylether diethylene glycol solution) is added to 100 μl of 100 mM phosphate buffer (pH 7.0) containing a NADPH production system (100 μM NADP, 100 μM glucose-6-phosphate, 1 unit of glucose-6-phosphate dehydrogenase), TcOYE and an inhibitor, and is allowed to react at 37° C. for two minutes. For reaction under anaerobic condition, after bubbling the reaction mixture with argon gas for five minutes, 100 μM NADPH or NADH and 1 μl PGH₂ solution are added, and the mixture is allowed to react in argon gas at 37° C. for two minutes.

250 μl of a stop solution (diethyl ether:methanol:2M citric acid (30:4:1 mixed solution)) at −20° C. and an excess amount of anhydrous sodium sulfate are added to stop the reaction, and the remaining substrate ([1-14C]PGH2) and the enzymatic reaction product ([1-14C]PGF_{2α}) are extracted in ether at the same time. Part (about 50 μl) of the ether layer is applied onto a silica gel thin layer (Merck & Co.) in a low-temperature room, and thin-layer chromatography (eluent:diethyl ether:methanol:acetic acid (90:2:1 mixed solution)) is performed in a refrigerated room at −20° C. After development, autoradiography of the thin layer plate is performed using the image analyzer FL2000 (Fuji Photo Film), and the substrate to product ratio is calculated to determine enzymatic activity.

When non-labeled PGH2 is used, the substrate and a product after reaction are determined by separation using LC-MS (liquid chromatography-mass spectrometry; Waters Alliance LC-MS system, 2690 separation module, 996 photodiode array detector, ZQ4000 mass detector; Inertsil-ODS3 column), and the substrate to product ratio is calculated similarly to determine enzymatic activity.

The enzymatic reaction described above is conducted in the presence or absence of an inhibitor and a decrease in the reaction rate due to the inhibitor is examined to determine whether or not the reduction is inhibited. Because it is very likely that Trypanosoma cruzi uses prostaglandin to sustain parasitism in host cells, an inhibitor of TcOYE may be used to inhibit Trypanosoma cruzi parasitism in host cells.

The present invention further relates to a method of screening trypanocidal drugs against Trypanosoma cruzi infection, comprising the steps of:

(i) incubating the protein TcOYE with a candidate compound in the presence of NADPH or NADH, and

(ii) determining whether or not the compound produces radicals as a result of one-electron reduction by the protein.

One milliliter of 100 mM phosphate buffer (pH 7.0) containing TcOYE and a substrate candidate is bubbled with argon gas for five minutes to make an anaerobic environment. Then, 100 μM NADPH or NADH is added to the reaction mixture and allowed to react at 37° C. under anaerobic condition, and the decrease in NADPH or NADH is followed by determining absorbance at 340 nm.

Generation of radicals as a result of one-electron reduction is determined as follows. A hundred microliter of 5 mM tris-HCl buffer (pH 7.0) containing TcOYE and a substrate candidate is displaced with argon gas for 5 minutes to make an anaerobic environment. Then, 10 mM NADPH or NADH is added to the reaction mixture and allowed to react at 25° C. or 37° C. for 3 minutes under anaerobic condition. Part of the reaction mixture is analyzed with an electron spin resonance spectrometer (JEOL X-band spectrometer) to determine the generation of radicals. The measurement conditions and analytical method are described in the paper below. It is believed that the radicals produced react with oxygen to generate superoxide anion radicals (Moreno S. N. J. et al., J. Biol. Chem. 259: 6298-6305, 1984), which kill Trypanosoma cruzi.

The present invention further relates to a method of diagnosing Trypanosoma cruzi infection, comprising the steps of:

(i) incubating a specimen or the extract of a specimen with the anti-TcOYE antibody described above, and

(ii) examining whether or not an antigen/antibody complex forms.

Examples of specimens used for diagnosis include blood samples from suspected patients with Trypanosoma cruzi infection, biopsies such as muscular tissues, and body fluids such as cerebrospinal fluid. The term “extract of a specimen” refers to protein, DNA or RNA which is extracted from the specimen described above.

The specimen described above is allowed to react with hyposmotic buffer solution to extract TcOYE from infecting Trypanosoma cruzi. The extract is allowed to react with an anti-TcOYE antibody to determine whether or not an antigen/antibody complex forms.

In addition, tissue sections and smears may be allowed to react with an anti-TcOYE antibody, then with a suitable fluorescent material or an enzyme-labeled secondary antibody to visualize localization of Trypanosoma cruzi.

Ordinary western blotting, the ELISA method using an immobilized antibody or the latex coagulating method can be used for the detection of an antigen-antibody complex. The immunohistochemical stain method commonly used (enzyme antibody stain and fluorescent antibody stain) can be used for detection in tissue sections and smear samples.

The present invention further relates to a method of diagnosing Trypanosoma cruzi infection, comprising the steps of:

(i) incubating a specimen or the extract of a specimen with the gene encoding TcOYE or a fragment thereof, and

(ii) examining whether or not they hybridize to each other.

The DNA extracted from the specimen described above is digested with various restriction enzymes, and DNA fragments are separated by agarose gel electrophoresis and transcribed onto a nylon film. The transcribed film is allowed to react with a cDNA or RNA probe of TcOYE labeled with a radioisotope or digoxigenin to determine the presence of a gene binding the probe (southern blotting). Alternatively, RNA extracted from the specimen described above may be used to determine the presence of the mRNA of TcOYE in a similar manner (northern blotting).

For the detection of the TcOYE gene or mRNA in tissue sections and smears, the in situ hybridization method may be used with a cDNA or RNA probe of TcOYE which is labeled with a radioisotope or digoxigenin.

The present invention further relates to a method of diagnosing Trypanosoma cruzi infection, comprising the steps of:

(i) preparing DNA collected from a specimen or cDNA synthesized from the mRNA in a specimen using reverse transcriptase,

(ii) performing PCR using this DNA as template, and using the nucleotide sequence contained in the cDNA of TcOYE of SEQ ID NO: 1 as a sense primer and an antisense primer, and

(iii) examining whether or not the cDNA of TcOYE is amplified.

For example, a sense primer (for example, 5′-ATGGCGACGTTCCCTGAACTCC-3′) (SEQ ID NO: 8) and an antisense primer (for example, 5′-TTATTTGTTGTACGTCGGGTA-3′) (SEQ ID NO: 9) contained in cDNA of TcOYE may be used for polymerase chain reaction (PCR) using the DNA from a small amount of body fluids infected with Trypanosoma cruzi, such as whole blood, muscular tissue and cerebrospinal fluid, as template to determine whether or not the cDNA of TcOYE is amplified. The PCR method includes, for example, a cycle of 95° C. for 5 minutes for DNA denaturation, and 30 cycles for amplification each consisting of 95° C. for 1 minute for DNA denaturation, 56° C. for 30 seconds for primer binding and 72° C. for 1 minute for elongation by DNA polymerase.

The following examples further illustrate the present invention, but are not intended to limit the scope of the invention.

### EXAMPLES

### Example 1

Prostaglandin Synthesizing System in Trypanosoma cruzi

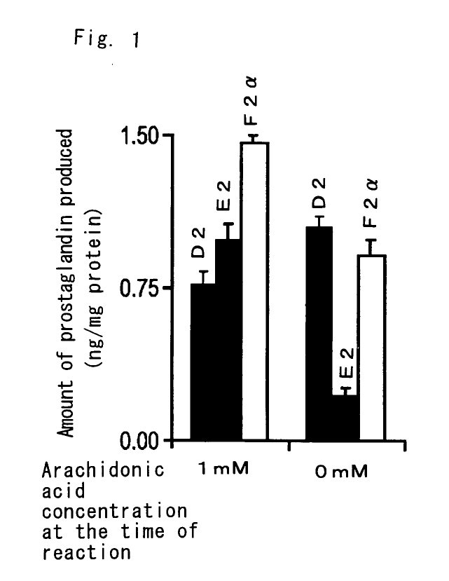

The vegetative form (epimastigote) of Trypanosoma cruzi YNIH strain in the insect body (obtained from the National Institute of Infectious Diseases (1-23-1, Toyama, Shinjuku-ku, Tokyo)) was incubated by the conventional method (Nozaki T. et al., J. Biol. Chem. 276: 6516-6523, 2001) using synthetic culture medium. The cultured protozoa was destroyed by hyposmotic treatment and allowed to react with arachidonic acid, and the PGs produced were extracted with an organic solvent and separated/purified by HPLC. Determination using a commercial kit (Kubata B. X. et al., J. Exp. Med. 188: 1197-1202, 1998) showed that the crude extract of Trypanosoma cruzi produced PGD₂, PGE₂ and PGF₂, actively (see FIG. 1). Production of these PGs was completely prevented by heat treatment at 100° C. for 20 minutes, but was unaffected by 3 μM Aspirin or 42 μM indomethacin, which completely inhibit PG production in mammals.

### Example 2

Prostaglandin H₂-F_{2α} Reductase Activity in Trypanosoma cruzi

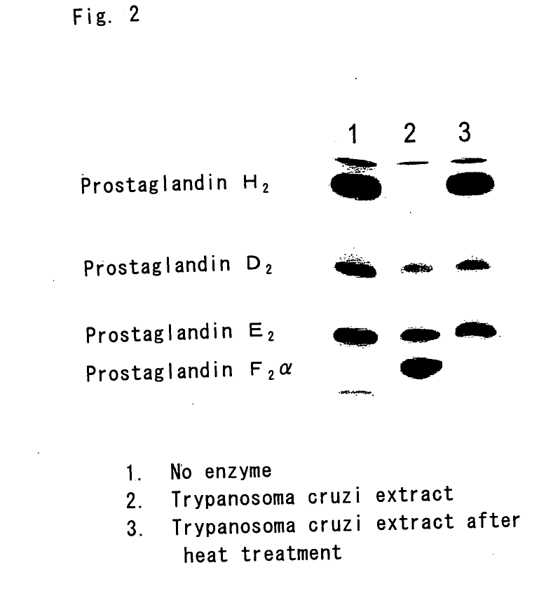

40 μM [1-14C]-PGH2 is allowed to react with 500 μM NADPH in 0.1M phosphate buffer (pH 7.0) undergoing argon gas bubbling at 37° C. for 2 minutes under anaerobic condition. If the soluble fraction of Trypanosoma cruzi is added, almost all PGH₂ is converted into PGF_{2α} (see FIG. 2). However, this conversion will not take place unless the heat-denatured soluble fraction of Trypanosoma cruzi or NADPH is added.

### Example 3

Purification of the Prostaglandin H2-F2α Reductase from Trypanosoma cruzi and Amino Acid Sequencing Thereof.

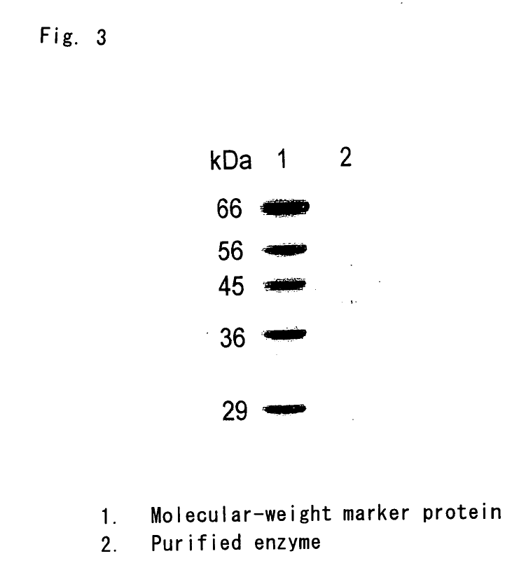

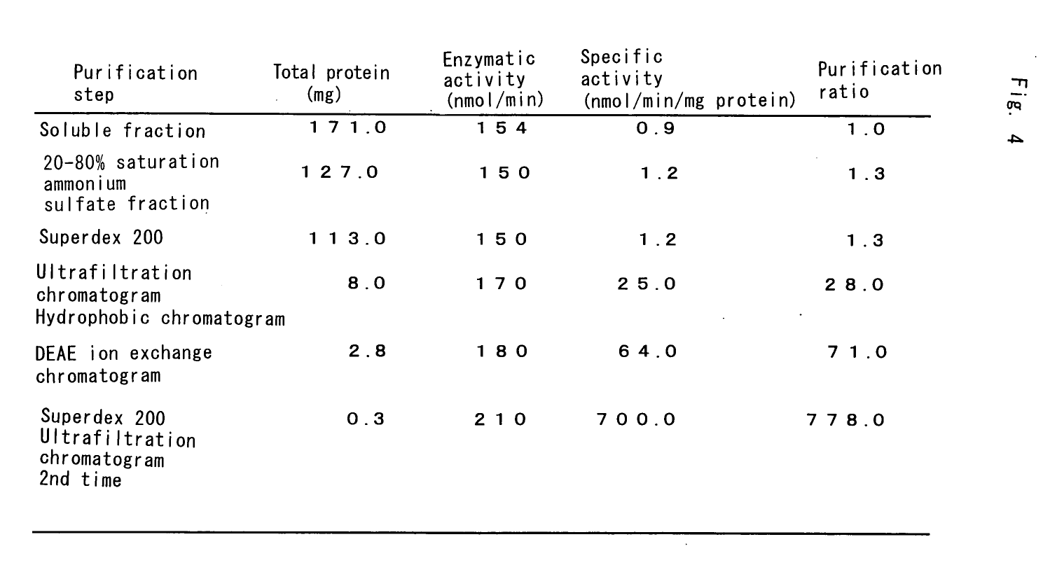

The soluble fraction of Trypanosoma cruzi was subjected to ammonium sulfate fractionation and the 20 to 80% ammonium sulfate saturation fractions were collected. These fractions were fractionated by gel filtration column chromatography (Hiload 16/60 Superdex 200 pg column, Amersham Pharmacia Biotec). The active fraction was concentrated with a Centricon concentrator (Millipore) with a cut-off value of 3,000 molecular weight, dialyzed in 20 mM phosphate buffer (pH 7.0), adsorbed by reversed phase column chromatography (Resource PHE reversed phase column, Amersham Pharmacia Biotec) equilibrated with 20 mM phosphate buffer (pH 7.0) containing 2 mol ammonium sulfate, and eluted with reverse-gradient ammonium sulfate, ranging from 2 mol to 0 mol, containing 0.1% Tween 20. The active fraction was dialyzed in 20 mM Tris-HCL buffer (pH 8.0), adsorbed to an ion exchange resin column (Hiprep 16/60 DEAE ion-exchange column, Amersham Pharmacia Biotec) equilibrated with this buffer and eluted in a linear concentration gradient of NaCl ranging from 0 to 400 mM. Gel filtration column chromatography (Hiload 16/60 Superdex 200 pg column, Amersham Pharmacia Biotec) of the active fraction resulted in a purified enzyme displaying a uniform band at molecular weight of 42,000 on SDS polyacrylamide gel electrophoresis, with a specific activity of about 700 nmol/min/mg protein and a yield in terms of PGH₂-F_{2α} reductase activity of about 1% with an about 1630-fold purification ratio (see FIGS. 3 and 4).

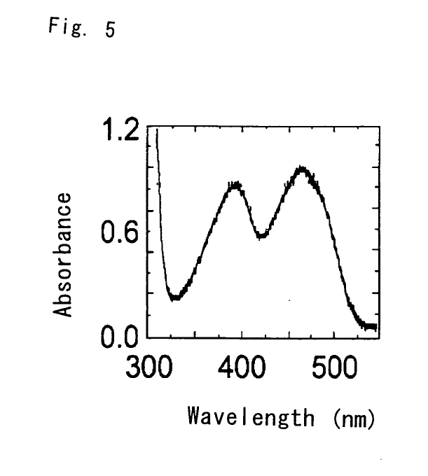

Purified PGH2-PGF2α reductase binds a yellow pigment. Its absorption spectrum in the visible range has an absorption maximum near 379 nm and 462 nm when in oxidation state. The visible absorption spectrum disappears when 100 μM NADPH is added to reduce the enzyme. Thus, the enzyme is a flavoprotein that binds one molecule of flavin mononucleotide (FMN) per molecule of the enzyme (see FIG. 5).

When the purified enzyme was treated with lysyl endopeptidase (Rosenfeld, J. et al., Anal. Biochem., 203: 173-179, 1992) and separated by reversed phase chromatography, three peptides were collected, whose amino acid sequences were determined as below.

Peptide 1: AsnArgIleIleMetAlaProLeuThrArg (SEQ ID NO: 3)

Peptide 2: AspHisArgIleProValTyrPheAlaAla (SEQ ID NO: 4)

Peptide 3: IleSerAsnLeuArgTyrAspPheGluGlu (SEQ ID NO: 5)

### Example 4

Expression of a Recombinant Protein Using cDNA Cloning of TcOYE and E. coli

Search of the EMBL/GenBank/DDBJ Database for the amino acid sequences of the three peptides obtained revealed two of the three peptides, and the remaining peptide with one different amino acid residue, in the predicted protein translation region of the 1,686 bp gene designated U31282 (Catmull, J. and Donelson, J. E., EMBL/GenBank/DDBJ Database, 1995, a homologous gene of the yeast old yellow enzyme gene found with the cytozoic type of Trypanosoma cruzi, described as a Trypanosoma cruzi reductase).

The base sequence below was synthesized from the nucleotide sequence of the protein translation region of Trypanosoma cruzi reductase as a sense primer to which an EcoRI restriction enzyme sequence was added at the 5′ end,

5′-CGGAATTCATGGCGACGTTCCCTGAACTTC-3′ (SEQ ID NO: 6) and the base sequence below as an antisense primer to which an XhoI restriction enzyme sequence was added at the 5′ end,

5′-CCGCTCGAGTTATTTGTTGTACGTCGGGTA-3′ (SEQ ID NO: 7).

Total RNA was extracted from the vegetative form of Trypanosoma cruzi in an insect body using the Guanidine hydrochloride-phenol method (ISOGEN solution, Nippon Gene) and annealed to an oligo dT-adapter primer (Takara Shuzo Co., Ltd.) to synthesize single-strand cDNA using avian myeloblastosis virus reverse transcriptase (Takara Shuzo Co., Ltd.). PCR using the synthetic sense and antisense primers described above resulted in amplification of the cDNA containing a protein translation region of a molecular weight of 42,260 comprising 379 amino acid residues (SEQ ID Nos. 1 and 2). The nucleotide sequence of the cDNA obtained differed from U31282 at 6 sites, one of which was accompanied by variation in amino acid residues. The entire amino acid sequences of the three peptides determined with the purified enzyme were contained in the protein translation region of the cDNA obtained.

The cDNA obtained was inserted into the EcoRI/XhoI site of the pGEX-4T-1 vector (Amersham Pharmacia Biotec) to produce a protein expression vector.

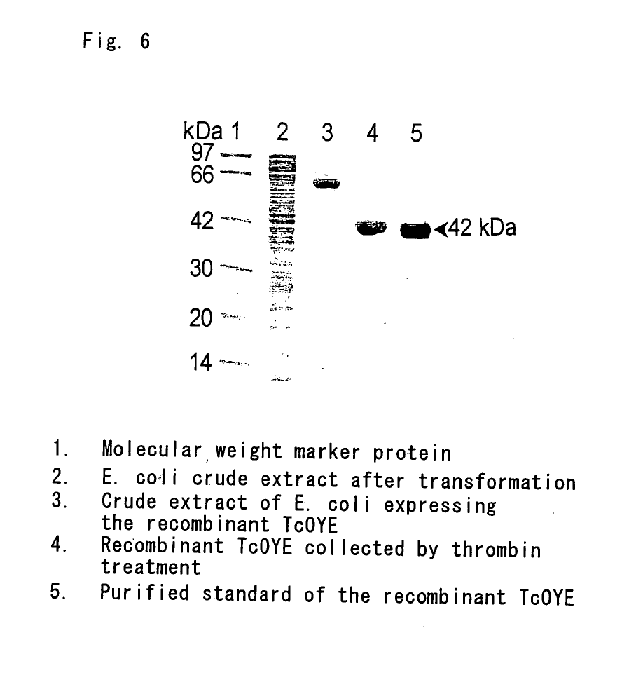

When E. coli BL21 was transformed with this vector and incubated for 7 hours in the presence of 0.5 mM isopropyl-β-D-thiogalactosylpyranoside, the recombinant TcOYE was expressed in the E. coli soluble fraction as a fusion protein with glutathione transferase. The E. coli cells were destroyed ultrasonically and the extract was subjected to a glutathione affinity column (glutathione Sepharose 4B, Amersham Pharmacia Biotec) to adsorb the fusion protein to resin, and, after washing, the column was treated with thrombin to recover the recombinant TcOYE. Thus, highly purified recombinant TcOYE was produced easily in large quantities (see FIG. 6).

### Example 5

Substrate Specificity of the Reduction Reaction by the Recombinant TcOYE

The PGH₂-F_{2α} reductase activity of the purified recombinant TcOYE was 766 nmol/minute/mg protein, a value representing a specific activity almost similar to the specimen purified from the soluble fraction of Trypanosoma cruzi.

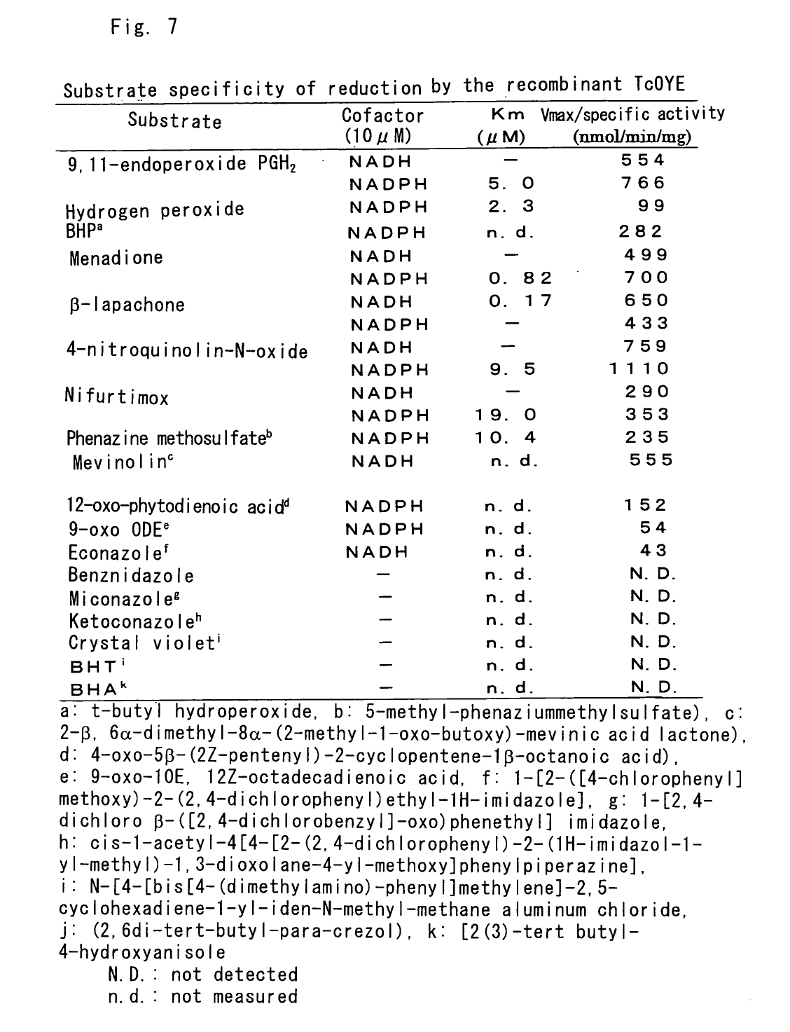

The recombinant TcOYE was allowed to react with 500 μM NADPH or NADH in argon-bubbled 0.1 M phosphate buffer (pH 7.0) and the decrease in absorption at 340 nm was measured to determine substrate specificity for various compounds; results showed that TcOYE reduced hydrogen peroxide and butyl peroxide (see FIG. 7). TcOYE also reduces anti-Trypanosomal quinones and nitro compounds, such as menadion, β-lapachone, 4-nitroquinoline-4-oxide, nifurtimox, phenazine methosulfate (5-methyl-phenaziummethylsulfate), mevinolin (2β,6α-dimethyl-8α-(2-methyl-1-oxo-butoxy)-mevinic acid lactone), 12-oxo-phytodienoic acid (4-oxo-5β-(2Z-pentenyl)-2-cyclopentene-1β-octanoic acid), 9-oxo-10E,12Z-octadecadienoic acid, and econazole (1-[2 ([4-chlorophenyl]methoxy)-2-(2,4-dichlorophenyl)eth yl-1H-imidazole)]. However, it did not reduce benznidazole or crystal violet (N-[4[bis[4-(dichlorophenyl)-2-(1H-imidazol-yl-methyl)-1,3-dioxolane-4-yl-methoxy]phenyl]piperazine]) (see FIG. 7).

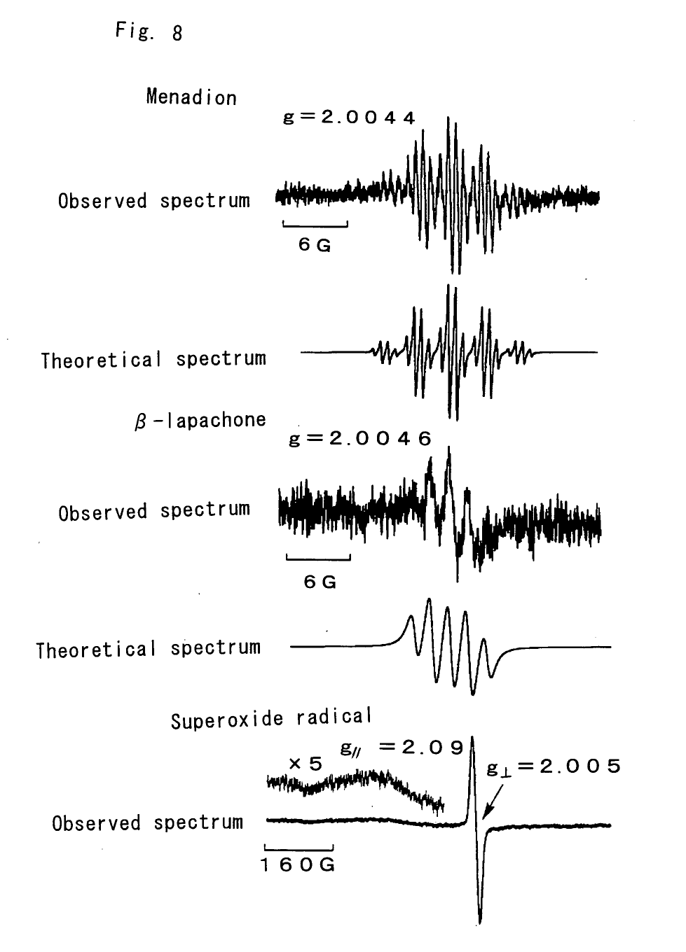

The electron spin resonance spectrum of the reaction product of TcOYE was measured with a JEOL X-band spectrometer (JEOL Ltd.) (Moreno, S. N. J. et al., J. Biol. Chem. 259: 6298-6305, 1984); a signal was detected that showed that naphtoquinone compounds such as menadion and β-lapachone underwent one-electron reduction and produced semiquinone radicals. The semiquinone radicals reacted with oxygen and produced superoxide anion radicals, which kill the protozoa (see FIG. 8). On the other hand, nitro heterocyclic compounds, such as nifurtimox, 4-nitroquinoline-N-oxide or mevinolin, underwent two-electron reduction and did not produce radicals.

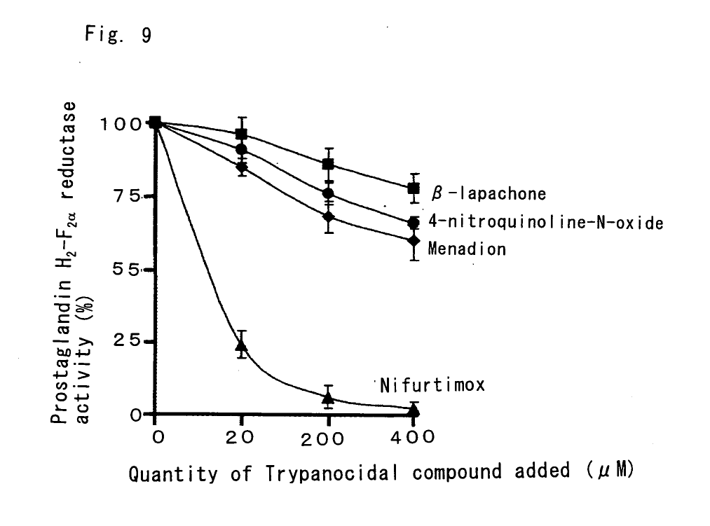

Naphtoquinone compounds and nitro heterocyclic compounds that are reduced by TcOYE inhibit the PGH₂-F_{2α} reducing activity of TcOYE in a dose-dependent manner. Nifurtimox inhibits the activity most strongly and the inhibiting effect of menadion, β-lapachone and 4-nitroquinoline-N-oxide is weak (see FIG. 9).

### Example 6

Production of Anti-TcOYE Antibody

Antigen of the recombinant TcOYE (300 μg) dissolved in 5 mM Tris-HCl buffer (pH 8.0) was mixed with an equal quantity of Freund complete adjuvant (Difco) and emulsified. The antigen emulsion was administered to a female Japanese white rabbit Kb1 at 20 subcutaneous sites in the shoulder for immunization. Then, every two weeks, an equal dose of the antigen emulsified by adding an equal quantity of incomplete Freund's adjuvant was administered a total of four times. Four weeks following the second immunization, blood was sampled from the rabbit's ear vein. The blood sample was allowed to set overnight at 4° C. and the serum was collected by centrifugal separation (1000×g, for 20 minutes). The blood serum was separated by protein A sepharose chromatography (Amersham Pharmacia Biotech), and the IgG fraction was purified.

### Example 7

Anti-TcOYE Antibody and Immunoabsorption Test of Drug Metabolizing Activity Using the Same

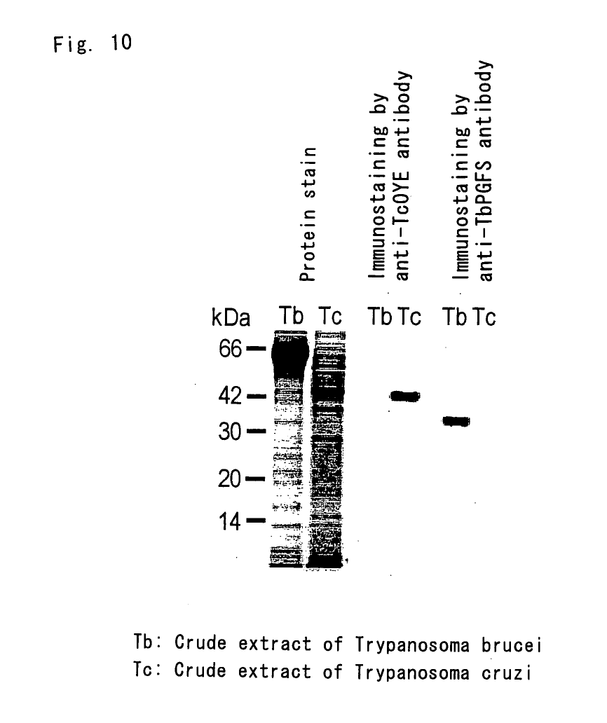

The antibody obtained by immunizing the rabbit with the purified recombinant TcOYE displayed immunological cross-reaction with only TcOYE in the Western blot assay using the crude extract of Trypanosoma cruzi. No protein was detected in the crude extract of Trypanosoma bruci or leishmania that exhibited immunological cross-reaction or bound TbPGFS. On the contrary, the antibody obtained by immunizing a rabbit with the recombinant TbPGFS did not recognize TcOYE, and no protein displaying immunological cross-reaction was detected in the crude extract of Trypanosoma cruzi (see FIG. 10).

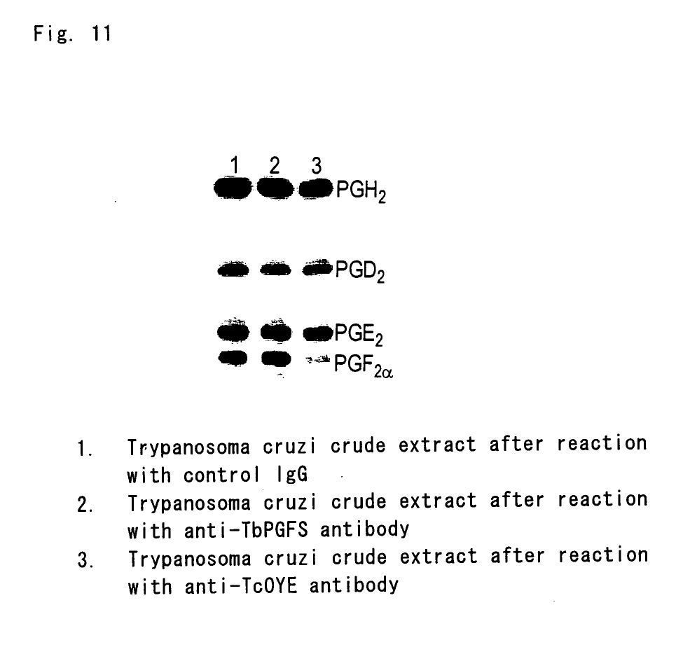

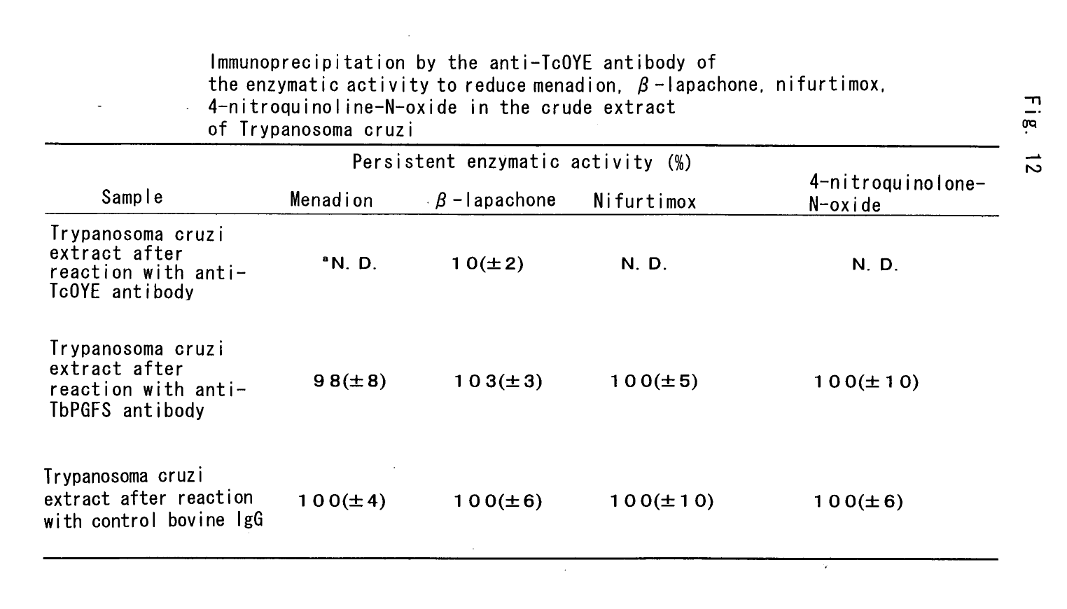

In an immunoabsorption test using the crude extract of Trypanosoma cruzi, the anti-TcOYE antibody absorbed the PGH2-F2α reductase activity almost completely, but the anti-TbPGFS antibody had no effect on the activity (see FIG. 11). The anti-TcOYE antibody almost completely immunoprecipitated the enzymatic activity to reduce menadion, β-lapachone, nifurtimox, 4-nitroquinoline-N-oxide in the crude extract of Trypanosoma cruzi (see FIG. 12).

These results show that the activity of the PGH₂-PGF_{2α} reductase and the activity to reduce such compounds as menadione, β-lapachone, nifurtimox and 4-nitroquinoline-N-oxide in the crude extract of Trypanosoma cruzi are mediated by TcOYE for the most part.

1. A flavoprotein (TcOYE) derived from Trypanosoma cruzi, having an enzymatic activity to reduce prostaglandin H₂ to prostaglandin F_{2α}.

2. A recombinant protein of (a), (b) or (c) below:
(a) a protein comprising the amino acid sequence represented by SEQ ID NO: 2
(b) a protein (hereinafter sometimes referred to as “variant TcOYE”) comprising an amino acid sequence having deletion, substitution or addition of one or more amino acids in the amino acid sequence represented by SEQ ID No: 2, and having an enzymatic activity to reduce prostaglandin H₂ to prostaglandin F_{2α}, and
(c) a protein comprising a fragment of the amino acid sequence represented by SEQ ID NO: 2, and having an enzymatic activity to reduce prostaglandin H₂ to prostaglandin F_{2α}.

3. A gene encoding the protein according to claim 1 or 2.

4. The gene according to claim 3 comprising DNA comprising the base sequence of SEQ ID NO: 1.

5. An antibody to the protein according to claim 1 or 2.

6. A method of screening trypanocidal drugs for Trypanosoma cruzi infection, comprising the steps of:
(i) preparing the protein according to claim 1 or 2 and prostaglandin H₂,
(ii) incubating the protein and prostaglandin H₂ with a candidate compound in the presence of NADPH or NADH, and
(iii) examining whether or not reduction of prostaglandin H₂ to prostaglandin F_{2α} is inhibited.

7. A method of screening trypanocidal drugs for Trypanosoma cruzi infection, comprising the steps of:
(i) incubating the protein according to claim 1 or 2 with a candidate compound in the presence of NADPH or NADH, and
(ii) determining whether or not the compound produces radicals as a result of one-electron reduction by the protein.

8. A method of diagnosing Trypanosoma cruzi infection, comprising the steps of:
(i) incubating a specimen or the extract of a specimen with the antibody according to claim 5, and
(ii) examining whether or not an antigen/antibody complex forms.

9. A method of diagnosing Trypanosoma cruzi infection, comprising the steps of:
(i) incubating a specimen or the extract of a specimen with the gene according to claim 3 or a fragment thereof, and
(ii) examining whether or not the specimen or the extract of the specimen hybridizes with the gene or a fragment thereof.

10. A method of diagnosing Trypanosoma cruzi infection, comprising the steps of:
(i) preparing DNA collected from a specimen or cDNA by synthesis from RNA in a specimen using reverse transcriptase,
(ii) performing PCR using said DNA or said cDNA as a template, and using the nucleotide sequence contained in the cDNA of TcOYE of SEQ ID NO: 1 as a sense primer and an antisense primer, and
(iii) examining whether or not the cDNA of TcOYE is amplified.
